(19) United States
(12) Patent Application Publication
(10) Pub. No.: US 2025/0170121 A1
(43) Pub. Date: May 29, 2025

(54) ISOQUINOLINONE COMPOUND, AND PREPARATION METHOD AND USE THEREFOR

(71) Applicants: FUDAN UNIVERSITY, Shanghai (CN); SHANGHAI INSTITUTE OF MATERIA MEDICA , CHINESE ACADEMY OF SCIENCES, Shanghai (CN)

(72) Inventors: Yingxia LI, Shanghai (CN); Heyao WANG, Shanghai (CN); Yulong HE, Shanghai (CN); Shunyi LI, Shanghai (CN)

(21) Appl. No.: 18/841,895

(22) PCT Filed: Mar. 27, 2023
(86) PCT No.: PCT/CN2023/083988

§ 371 Date: Aug. 27, 2024

(30) Foreign Application Priority Data
Feb. 28, 2022 (CN) 202210191835.4

(51) Int. Cl. A61K 31/4725; A61P 3/00; C07D 217/26; C07D 401/04; C07D 405/04; C07D 405/10

(52) U.S. Cl. CPC A61K 31/4725; A61P 3/00; C07D 217/26; C07D 401/04; C07D 405/04; C07D 405/10

(57) ABSTRACT

Provided are an isoquinolinone compound as shown in formula (I), a pharmaceutically acceptable salt thereof, a prodrug molecule, and a mixture thereof, a preparation method therefor, a pharmaceutical composition containing the compound, and a use thereof as an FABP4/FABP5 dual-targeting inhibitor. Related aryl carboxylic acid compounds can be used for preparing drugs for treating metabolic diseases (such as diabetes, insulin resistance, hyperlipidemia, and atherosclerosis), autoimmune diseases and cancers.

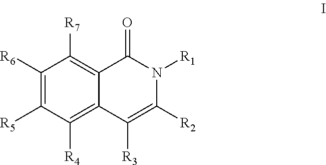

### TECHNICAL FIELD

The present invention belongs to the technical field of pharmaceutical chemistry and relates to fatty acid binding protein (FABP) 4 and/or 5 inhibitors, and specifically to novel FABP4/5inhibitors with an isoquinolinone structure. The present invention also relates to a method for preparing such compounds and their medical use as FABP4/5 inhibitors.

### BACKGROUND

With the development and improvement of human medical level, many ancient infectious diseases in the world have been successfully cured, while non-communicable diseases have become the main cause of morbidity and mortality in many countries in the world. Among non-communicable diseases, metabolic syndrome is becoming a public health problem worldwide. Metabolic syndrome refers to a group of metabolic disorders, namely obesity, diabetes, insulin resistance, hyperlipidemia, hyperglycemia and hypertension. These factors directly increase the incidence and mortality of cardiovascular diseases.

At present, the etiology, molecular and cellular mechanisms of metabolic syndrome have not been fully elucidated. Obesity-related chronic inflammation has been identified as the pathophysiological basis of diabetes and metabolic syndrome. In 2014, a study by the World Health Organization showed that more than 1.9 billion adults were overweight: more than 600 million of them suffered from obesity, which means that 39% of adults were overweight and 13% were obese. In addition, 41 million children under the age of 5 were overweight or obese. Obesity, especially the expansion of visceral adipose tissue, can cause the body to be in a “chronic low-grade inflammatory state”, referred to as metabolic inflammation, which is characterized by dysregulation of adipose factor secretion. Studies have found that adipokines, fatty acid binding protein 4 (FABP4) and fatty acid binding protein 5 (FABP5), which are expressed in both adipocytes and macrophages, play an important role in metabolic diseases such as insulin resistance and atherosclerosis.

The occurrence and development of metabolic diseases are closely related to abnormal lipid metabolism in the body. Free fatty acids, as important lipid energy sources and signaling molecules in cells, participate in regulating various enzyme catalytic reactions and signal transduction in cells after being transported to relevant action sites, thereby maintaining the body's metabolic balance. Fatty acid binding proteins (FABPs) are a class of lipid chaperone proteins that play an important role in the uptake, transportation and metabolic regulation of fatty acids in the body, affecting various biological processes in cells.

Fatty acid binding protein 4 (FABP4, also known as aP2) is a member of the intracellular lipid chaperone protein-fatty acid binding protein family, which is mainly expressed in adipocytes, macrophages and endothelial cells. In cells, FABP4, by acting on multiple signaling pathways, regulates lipid metabolism and inflammatory pathways in cells, weakens the function of insulin, promotes glucose production, and reduces cholesterol efflux, which is one of the pathogenesis of metabolic diseases such as diabetes and atherosclerosis. In addition to functioning in cells, adipocytes can also secrete FABP4 into the blood circulation, resulting in a variety of biological effects. Clinical studies have shown that the level of

FABP4 in plasma of different races is closely related to a variety of diseases. High expression level of FABP4 in blood can promote the development of insulin resistance, diabetes, atherosclerosis, hypertension and cardiac dysfunction, leading to poor prognosis of cardiovascular disease. In addition, sympathetic nervous system activation mediated by glucose and lipid metabolism disorders and cardiovascular diseases and upregulation of inflammatory cytokine expression may promote lipolysis in adipocytes, leading to a vicious cycle caused by increased FABP4 secretion.

Fatty acid binding protein 5 is also known as epidermal fatty acid binding protein (E-FABP), psoriasis-associated fatty acid binding protein (PA-FABP) or mal 1. FABP5 is mainly distributed in epidermal cells and is also expressed in adipocytes, macrophages and dendritic cells. FABP5 and FABP4 have 52% amino acid sequence homology and similar affinity and selectivity for fatty acid ligands, suggesting that FABP5 has a synergistic effect on FABP4 in exerting biological functions. Under normal conditions, the content of FABP4 in adipocytes is 100 times that of FABP5, but the expression of FABP5 will increase compensatorily in FABP4 knockout adipocytes. Studies have shown that FABP5 knockout adipocytes significantly increase glucose uptake under insulin stimulation: FABP5 knockout normal mice have significantly decreased plasma cholesterol and triglycerides: similar to FABP4, secretome analysis shows that adipocytes can also secrete FABP5, and exogenous FABP4 and FABP5 can regulate the transcription and metabolic functions of adipose tissue-derived stem cells. Clinical studies have shown that FABP5 promotes the progression of atherosclerosis by inhibiting the cholesterol efflux function of macrophages and can be used as a biomarker for predicting atherosclerosis. As a FABP4-related lipid protein, FABP5 expressed in adipocytes and macrophages also plays an important role in metabolic diseases such as insulin resistance, diabetes and arteriosclerosis.

Studies on FABP4 knockout mouse models have found that FABP4 plays an important role in metabolic syndrome. FABP4 knockout mice showed significantly improved hyperinsulinemia and insulin resistance induced by high fat diet, and blood sugar in mice was effectively controlled and inflammation in adipose tissue was alleviated. Recent studies have shown that total knockout or tissue or organ specific knockout of FABP4 in macrophages could significantly reduce atherosclerotic plaques in apolipoprotein E-deficient mice model. In addition, in FABP4 knockout mice, the expression of inflammatory cytokines including tumor necrosis factor-a (TNF-a), interleukin-6 (IL-6) and monocyte chemoattractant protein-1 (MCP-1) was strongly inhibited. Under high-fat diet conditions, knockout of FABP5 could improve oral glucose tolerance and insulin sensitivity in obese mice, and the levels of insulin and blood glucose in plasma were lower than those in the control group: while overexpression of FABP5 could impair glucose tolerance in mice. On the other hand, after 8 weeks of high-fat diet feeding, the atherosclerotic lesions of FABP5/low-density lipoprotein receptor (LDLR--) double knockout mice were reduced by 36% compared with the control group, the aggregation of macrophages in lesion plaques was reduced, and the expression of inflammatory factors, interleukin-6 (IL-6) and cyclooxygenase 2 (COX-2) was significantly decreased.

On the other hand, FABP4/5 double mutant (FABP4-5^{-/-}) mice could avoid high-fat diet-induced obesity, and their improvement in insulin sensitivity and glucose homeostasis was significantly better than the phenotype of single FABP4/5 knockout. In addition, Fabp4-54-mice could avoid high-fat diet-induced hepatic steatosis, enhance muscle AMPK kinase activity and reduce the expression of key fatty acid metabolism enzyme Scd1 in the liver, thereby reducing lipid accumulation. In the leptin gene-deficient (ob/ob^{-/-}) mouse model, the simultaneous knockout of FABP4/5 could also improve the glucose tolerance and insulin sensitivity of mice, which was better than the mouse model with single knockout, and inhibit the activity of stearoyl desaturase SCD-1 in the liver, thus inhibiting the formation of fatty liver in mice. In the apoE-mutant mouse model, the combined knockout of FABP4/5 could significantly inhibit the degree of atherosclerotic lesions in mice, improve the abnormal glucose and lipid metabolism of mice and prolong the survival of apoE^{-/-} mutant mice. In summary, FABP4/5 dual-target inhibitors can more effectively treat metabolic diseases such as type 2 diabetes and atherosclerosis, as well as autoimmune diseases such as psoriasis and rheumatoid arthritis.

Adipose-derived adipokines—FABP4 and FABP5—play an important role in the initiation, development, and metastasis of cancer. FABP4 promotes tumor cell proliferation and metastasis through lipid transport and induction of fatty acid oxidation, and plays an active role in the mutual communication between cancer cells and adipose tissue. In malignant gliomas, overexpressed FABP4 can regulate vascular endothelial growth factor and fibroblast growth factor to promote angiogenesis in tumor tissue. Exogenous FABP4 can promote prostate cancer proliferation. Treatment of prostate cancer cells with the selective FABP4 inhibitor BMS309403 can inhibit cell proliferation and metastasis and promote cell apoptosis. In breast cancer cells, exogenous FABP4 can activate Akt and MAPK signaling pathways to promote cell proliferation. Epidemiological studies have shown that the level of FABP4 in the serum of breast cancer patients is significantly higher than that of healthy controls. These high-expressed FABP4s are positively correlated with tumor size and lymph node involvement. The expression of FABP4 and FABP3 is upregulated in patients with non-small cell lung cancer. The high expression of FABP4 and FABP3 is associated with the grade of non-small cell lung cancer and patient survival. FABP5 may inhibit gastric cancer proliferation, apoptosis and invasion by regulating fatty acid metabolism cell signaling pathways. Knockout of FABP5 can reduce invasion, proliferation and cell growth through G0/G1 cell cycle arrest and increase apoptosis of gastric cancer cells via changes in gene expression. Overexpression of FABP5 in liver cancer tissues is associated with tumor occurrence, invasion and metastasis. Therefore, FABP5 can be used as an important marker and molecular target for liver cancer treatment strategies. In prostate cancer tissues, FABP5 is upregulated as a specific oncogene associated with metastasis, inducing cancer cell proliferation through nuclear receptors PPARβ/δ, and increasing angiogenesis in prostate cancer cells through the FABP5-PPARr-VEGF signaling transduction pathway, thereby promoting cancer cell metastasis. These studies show that FABP4 and FABP5 can be regarded as both new tumor-related clinical markers and new targets for the treatment of adipose tissue-related cancers.

A large number of FABP4, FABP5 and FABP4/5 small molecule inhibitors of different structural types have been developed and reported in some studies through means such as computer virtual screening, high-throughput screening or structure-based drug design, but so far, no small molecule inhibitors have entered clinical research. The reason may be related to the poor physicochemical properties, low selectivity (for FABP3) and poor pharmacokinetic properties of the compounds. Therefore, it is urgent to develop FABP4 inhibitors, FABP5inhibitors, especially FABP4/5 dual inhibitors with high selectivity, excellent physicochemical properties and high activities, for the treatment of metabolic diseases such as diabetes and atherosclerosis, autoimmune diseases such as psoriasis and rheumatoid arthritis, and malignant tumors such as breast cancer, gastric cancer and prostate cancer.

### SUMMARY OF THE INVENTION

The purpose of the present invention is to provide an isoquinolinone compound with FABP4/5 inhibitory activity, a pharmaceutically acceptable salt, stereoisomer, prodrug molecule thereof or a mixture thereof, and a preparation method, pharmaceutical composition and use thereof, especially for the treatment and/or prevention of metabolic diseases, inflammation, cancer and other diseases closely associated with FABP4/5.

In the first aspect, the present invention provides an isoquinolinone compound represented by a general formula (I), a pharmaceutically acceptable salt, stereoisomer (such as enantiomer, diastereomer or racemate), prodrug molecule thereof or a mixture thereof:

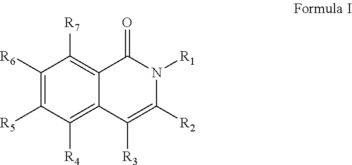

Wherein,

R₁ is selected from the group consisting of substituted or unsubstituted phenyl, C₅-C₁₂ aromatic heterocyclyl containing oxygen, nitrogen and/or sulfur, C₃-C₈ cycloalkyl, and 4-7 membered heterocyclyl containing 1 to 3 heteroatoms of oxygen, nitrogen and/or sulfur; in the case where the R₁ group has a substituent (i.e. “substituted”), each substituent is independently selected from the group consisting of halogen, hydroxyl, hydroxymethyl, thiol, amino, cyano, nitro, carboxyl, ester group, trifluoromethyl, trifluoromethoxy, C₁-C₆ straight-chain or branched-chain alkyl, C₁-C₆ straight-chain or branched-chain haloalkyl, C₁-C₆ straight-chain or branched-chain alkoxy, C₁-C₆ straight-chain or branched-chain haloalkoxy, C₁-C₆ straight-chain or branched-chain alkylcarbonyloxy, and C₁-C₆ straight-chain or branched-chain hydroxyalkyl:

R₂ is selected from the group consisting of carboxyl, cyano, hydroxyl, sulfonyl hydroxide, sulfonamide group, amide group, tetrazole group, squaryl, phosphate group, hydroxamic acid group, and hydroxyisoxazole group:

R₃ is selected from the group consisting of substituted or unsubstituted phenyl: C₆-C₁₂ aryl: benzyl; C₅-C₇ aromatic heterocycle containing oxygen, nitrogen and sulfur: C₃-C₁₄ cycloalkyl: and 4-8 membered heterocyclyl: in the case where the R₃ group has a substituent (i.e. “substituted”), each substituent is independently selected from the group consisting of halogen, hydroxyl, hydroxymethyl, thiol, amino, cyano, nitro, carboxyl, ester group, trifluoromethyl, trifluoromethoxy, C₁-C₆ straight-chain alkyl or C₃-C₆ branched-chain alkyl, C₁-C₆ straight-chain haloalkyl or C₃-C₆ branched-chain haloalkyl, C₁-C₆ straight-chain alkoxy or C₃-C₆ branched-chain alkoxy, and C₁-C₆ straight-chain haloalkoxy:

R₄ to R₇ are each independently selected from the group consisting of hydrogen, halogen, hydroxyl, amino, cyano, thiol, trifluoromethyl, trifluoromethoxy, nitro, amide group, sulfonamide group, C₁-C₃ straight-chain or branched alkyl, and C₁-C₃ straight-chain or branched-chain alkoxy:

In certain preferred embodiments, in the compound represented by formula I of the present invention, its pharmaceutically acceptable salt, stereoisomer or prodrug molecule or mixture thereof:

R₁ is selected from the group consisting of C₃-C₈ cycloalkyl, 4-7 membered heterocyclyl containing 1 to 3 oxygen, nitrogen and/or sulfur, and substituted or unsubstituted phenyl: in the case where the R₁ group has a substituent (i.e. “substituted”), each substituent is independently selected from the group consisting of halogen, hydroxyl, hydroxymethyl, thiol, amino, cyano, nitro, carboxyl, ester group, trifluoromethyl, trifluoromethoxy, C₁-C₃ straight-chain or branched-chain alkyl, and C₁-C₃ straight-chain or branched-chain alkoxy:

R₂ is selected from the group consisting of carboxyl, sulfonyl hydroxide, sulfonamide group, amide group, tetrazole group, squaryl, and hydroxyisoxazole group:

R₃ is selected from the group consisting of substituted or unsubstituted phenyl: C₅-C₇ aromatic heterocycle containing oxygen, nitrogen and sulfur: C₃-C₁₄ cycloalkyl; and 4-8 membered heterocyclyl: in the case where the R3 group has a substituent (i.e. “substituted”), each substituent is independently selected from the group consisting of halogen, hydroxyl, hydroxymethyl, thiol, amino, cyano, nitro, carboxyl, ester group, trifluoromethyl, and trifluoromethoxy:

R₄ to R₇ are each independently selected from the group consisting of hydrogen, halogen, hydroxyl, amino, trifluoromethyl, trifluoromethoxy, nitro, and methoxy.

In some other preferred embodiments, R₅ is as defined above, R₂ is —COOH, R₃ is phenyl, R₄, R₆, and R₇ are independently hydrogen or they are simultaneously hydrogen.

In yet some other preferred embodiments, R₅ is as defined above, R₂ is —COOH, R₃ is substituted phenyl or aromatic heterocyclyl, R₄, R₆, and R₇ are independently hydrogen or they are simultaneously hydrogen.

In other embodiments, R₁ is selected from the group consisting of cyclohexyl, methylcyclohexyl, cyclopentyl, tetrahydropyranyl, cyclopropyl, cyclobutyl, cycloheptyl, chlorophenyl, piperidinyl and cyclopentyl: R₂ is carboxyl: R₃ is selected from the group consisting of phenyl, fluorophenyl, hydroxyphenyl, hydroxymethylphenyl, chlorofluorophenyl, trifluoromethylphenyl, dihydrobenzofuranyl, pyridinyl, and dimethylpyrazolyl: R₄ and R₇ are independently H or they are simultaneously H, R₅ is Cl, Br or methyl; and R₆ is H or Cl.

The C₅-C₁₂ aromatic heterocyclyl may be, for example, an aromatic heterocyclyl having 5, 6, 7, 8, 9, 10, 11 or 12 carbon atoms:

The C₃-C₈ cycloalkyl may be, for example, cyclopropyl, cyclobutyl, cyclopentyl or cyclohexyl:

The halogen may be, for example, F, Cl, Br or I.

The 4-7 membered heterocyclyl may be, for example, a 4-membered heterocyclyl, a 5-membered heterocyclyl, a 6-membered heterocyclyl or a 7-membered heterocyclyl having 1, 2 or 3 heteroatoms selected from O, N and S:

The C₁-C₆ straight-chain alkyl or C₁-C₆ branched-chain alkyl is a straight-chain alkyl with 1, 2, 3, 4, 5 or 6 carbon atoms or a C₃-C₆ branched-chain alkyl, for example, it can be methyl, ethyl, n-propyl, isopropyl, n-butyl, n-pentyl, n-hexyl, a branched butyl (such as sec-butyl or tert-butyl), a branched pentyl (isopentyl), a branched hexyl, etc.;

The above groups have the meanings commonly understood by those skilled in the art, for example:

The C₁-C₆ straight-chain haloalkyl may be a straight-chain haloalkyl having 1, 2, 3, 4, 5 or 6 carbon atoms and substituted with one or more (for example, 1, 2 or 3) halogen atoms:

The C₃-C₆ branched haloalkyl may be a branched haloalkyl having 3, 4, 5 or 6 carbon atoms and substituted with one or more (for example, 1, 2 or 3) halogen atoms:

The C₁-C₆ straight chain alkoxy is a straight chain alkoxy with 1, 2, 3, 4, 5 or 6 carbon atoms, such as methoxy, ethoxy, propoxy, butoxy, pentyloxy or butoxy, etc.;

The C₃-C₆ branched alkoxy is a branched alkoxy with 1, 2, 3, 4, 5 or 6 carbon atoms;

The C₆-C₁₂ aryl is an aryl with 6, 7, 8, 9, 10, 11 or 12 carbon atoms, such as phenyl, benzyl, phenethyl, etc.;

The C₅-C₇ aromatic heterocycle containing oxygen, nitrogen and sulfur may be, for example, an aromatic heterocycle having 5, 6 or 7 carbon atoms and containing one or more (for example, 1, 2 or 3) heteroatoms selected from oxygen, nitrogen and sulfur.

The C₃-C₁₄ cycloalkyl may be, for example, cyclopropyl, cyclobutyl, cyclopentyl, cyclohexyl, cycloheptyl, cyclooctyl, cyclononyl or cyclodecyl, etc.;

The 4-8 membered heterocyclyl may be, for example, a heterocyclyl having 4, 5, 6, 7 or 8 carbon atoms and containing a heteroatom such as O, N or S;

The C₁-C₃ straight chain alkyl or alkoxy may be, for example, methyl, methoxy, ethyl, ethoxy, propyl, propoxy, etc.

In some most preferred embodiments, the compound of the present invention or the pharmaceutically acceptable salt, stereoisomer, prodrug molecule thereof is selected from the following compounds Y1-Y22:

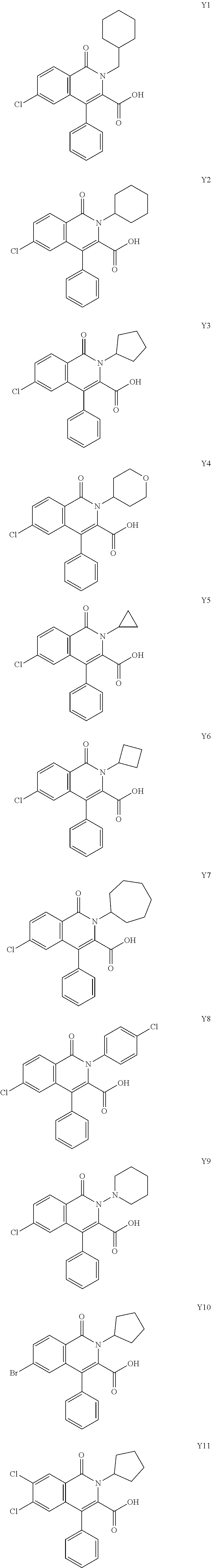

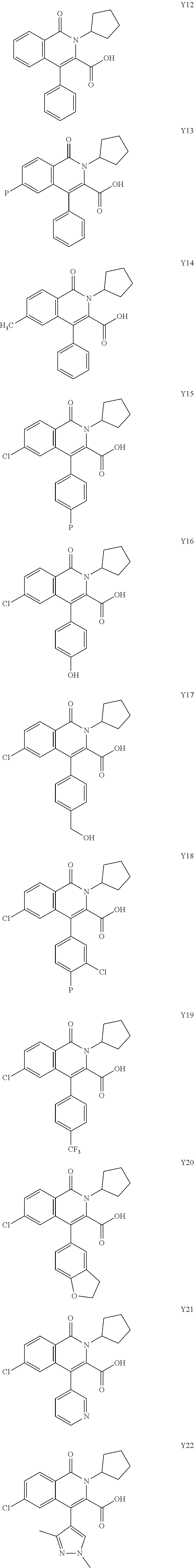

The pharmaceutically acceptable salt of the present invention may be a salt formed by an anion and a positively charged group on the compound of general formula I. Suitable anions may be one or more selected from the group consisting of chloride ion, bromide ion, iodide ion, sulfate, nitrate, phosphate, citrate, methanesulfonate, trifluoroacetate, acetate, malate, tartrate, fumarate, glutamate, glucuronate, lactate, glutarate, or maleate. Similarly, the pharmaceutically acceptable salt may be a salt formed by a cation and a negatively charged group on the compound of general formula I. Suitable cations may be one or more selected from the group consisting of sodium ion, potassium ion, magnesium ion, calcium ion, ammonium, and tetramethylammonium.

The compound of the present invention may also be a pharmaceutical composition in the form of an ester, a prodrug, or an N-oxide.

Another object of the present invention is to provide a use of the isoquinolinone compound of general formula I described in the first aspect of the present invention in the preparation of a drug for preventing or treating FABP4/5-mediated diseases.

The FABP4/5-mediated diseases may be, for example, metabolic diseases and cardiovascular and cerebrovascular diseases, including but not limited to: insulin resistance, metabolic syndrome, type 2 diabetes, hyperlipidemia, obesity, atherosclerosis, myocarditis, myocardial infarction, arrhythmia, coronary heart disease, hypertension, heart failure, myocardial hypertrophy, diabetic complications (including diabetic cardiomyopathy, diabetic nephropathy, diabetic ulcer, retinopathy and neuropathy, etc.), non-alcoholic fatty liver disease, non-alcoholic fatty hepatitis, cirrhosis, hyperuricemia, osteoporosis, polycystic ovary syndrome, etc.

The FABP4/5-mediated diseases, such as inflammatory diseases, autoimmune diseases, organ fibrosis diseases, neurological damage diseases or secondary diseases caused by pathogen infection, may include pneumonia, tuberculosis, inflammatory bowel disease, asthma, chronic obstructive pulmonary disease, chronic bronchitis, emphysema, bronchiolitis obliterans, allergic rhinitis, sinusitis, systemic lupus erythematosus, rheumatoid arthritis, osteoarthritis, pancreatitis, chronic nephritis, cystitis, multiple sclerosis, amyotrophic lateral sclerosis, etc.

The FABP4/5-mediated diseases, such as tumors, may include melanoma, lung cancer, breast cancer, gastric cancer, liver cancer, pancreatic cancer, colon cancer, kidney cancer, prostate cancer, bone cancer, lymphoma, mesenchymal cell carcinoma, acute myeloid leukemia, chronic myeloid leukemia, Hodgkin's lymphoma, glioma, astrocytoma, etc.

The present invention also provides a pharmaceutical composition for preventing or treating FABP4/5-mediated diseases, which contains a therapeutically effective amount of the isoquinolinone compound of general formula I, the pharmaceutically acceptable salt, stereoisomer, prodrug molecule or the mixture thereof as an active ingredient and a pharmaceutically acceptable excipient.

The excipients that can be mixed in the pharmaceutical composition of the present invention can be varied depending on the dosage forms, administration routes, etc. Excipients can include but are not limited to vehicles, adhesives, disintegrants, lubricants, flavoring agents, fragrant agents, colorants and/or sweeteners. The administration route of the pharmaceutical composition can be oral, sublingual, transdermal, intramuscular, subcutaneous, mucocutaneous or intravenous. The pharmaceutical composition can be in the form of a capsule, powder, tablet, granule, pill, injection, syrup, oral solution, inhalant, cream, ointment, suppository or patch, etc., which are conventionally used in pharmacy.

The preparation of the compound of the present invention can be carried out according to the following synthetic route or improved method.

### Synthesis Route 1

When R₂ is-COOH, R₃ is phenyl, R₄, R₆, and R₇ are hydrogen, and R₅ is as defined above, the synthesis route is as follows:

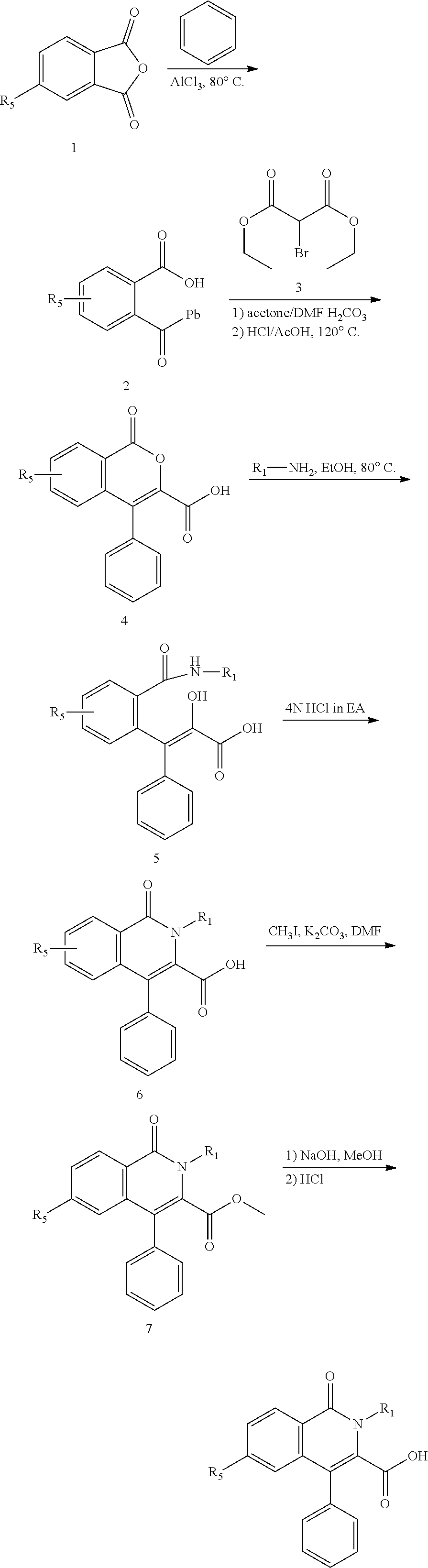

The starting material acid anhydride 1 is reacted with benzene to generate intermediate 2 through Friedel-Crafts reaction. Intermediate 2, a positional isomer, can be directly subjected to the next step without separation. Intermediate 2 is reacted with diethyl 2-bromomalonate in the presence of potassium carbonate/acetone to generate an intermediate ester, which is then cyclized under acidic conditions to generate Intermediate 4. Intermediate 4 is reacted with the raw material amine in ethanol through aminolysis to generate intermediate 5, which is then cyclized under acidic conditions to generate intermediate 6 (still a positional isomer). Intermediate 6 is then converted to its methyl ester in the presence of methanol/thionyl chloride. The target intermediate 7 is obtained by column chromatography and then hydrolyzed under alkaline conditions to generate the target compound.

### Synthesis Route 2

When R₂ is —COOH, R₃ is a substituted phenyl or aromatic heterocyclyl, R₄, R₆, and R₇ are hydrogen, and R₅ is as defined above, the synthesis route is as follows:

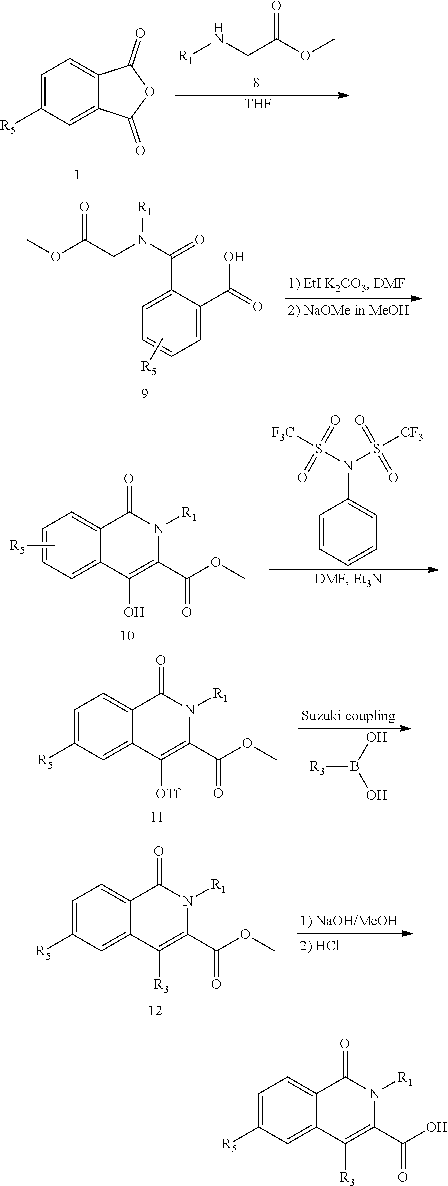

Starting material 1 and another raw material 8 are stirred in tetrahydrofuran solution at room temperature to undergo an aminolysis reaction to generate intermediate 9. Intermediate 9, a positional isomer, is directly subjected to the next step without purification. Intermediate 9 is then reacted with iodoethane to generate ethyl carboxylate, which is subjected to ester condensation under alkaline conditions to generate intermediate 10. Intermediate 10 is reacted with 1,1,1-trifluoro-N-phenyl-N-((trifluoromethyl)sulfonyl)methanesulfonamide in the presence of DMF/triethylamine to generate a key intermediate. After column chromatography purification and separation, the target intermediate 11 is obtained. Intermediate 11 is reacted with aromatic or aromatic heterocyclic boric acid esters with different substituents through suzuki reaction to generate intermediate 12, which is then hydrolyzed to generate the target compound under alkaline conditions.

The compound of the present invention can be administered alone or in combination with other therapeutic drugs (such as anti-tumor drugs).

### DESCRIPTION OF DRAWINGS

FIG. 1 shows the inhibitory effect of different compounds on lipolysis in 3T3-L1 cells, wherein the horizontal axis represents lipolysis in fold of FSK, Control represents blank control treatment, FSK represents forskolin treatment, BMS309403 represents positive control treatment, and Y2 and Y3 represent treatments with compounds Y2 and Y3 prepared in the present invention, respectively.

FIG. 2 shows the inhibitory effect of different compounds on MCP-1 secretion in THP-1 cells. In FIG. 2A to FIG. 2C, the vertical axis represents the amount (ng) of MCP-1 contained in each mg of cell protein: in FIG. 2D, the vertical axis represents the relative concentration of MCP-1 (ng/mg cellular protein % LPS). Wherein, Control represents blank control treatment, LPS represents lipopolysaccharide treatment, BMS309403 represents inhibitor positive control treatment, RO6806051 (RO) represents lead compound treatment, and Y2, Y3, Y15, Y16 and Y18 represent treatments with compounds Y2, Y3, Y15, Y16 and Y18 prepared in the present invention, respectively.

FIG. 3 shows the inhibitory effect of different compounds on IL-6 secretion in THP-1 cells. In FIG. 3, the vertical axis represents the amount (ng) of MCP-1 contained in each mg of cell protein. Wherein, Control (Con) represents blank control treatment, LPS represents lipopolysaccharide treatment, BMS309403 represents inhibitor positive control treatment, RO6806051 (RO) represents lead compound treatment, and Y2, Y3, Y15, Y16 and Y18 represent treatments with compounds Y2, Y3, Y15, Y16 and Y18 prepared in the present invention, respectively.

### DETAILS OF EMBODIMENTS

The content of the present invention is described in details with reference to the examples below. In the present invention, the following examples are provided for illustrating the present invention better and are not intended to limit the scope of the present invention. Various changes and modifications can be made to the present invention without departing from the spirit and scope of the present invention. The reagents used in the experimental methods described in the following examples are conventional reagents unless otherwise specified: the reagents and materials are commercially available unless otherwise specified.

### EXAMPLE 1

### 6-Chloro-2-cyclohexyl-1-oxo-4-phenyl-1,2-dihydroisoquinoline-3-carboxylic Acid (Y1)

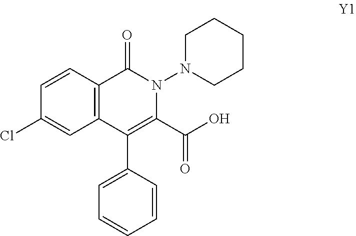 [SMILES: O=C(O)c1c(-c2ccccc2)c2cc(Cl)ccc2c(=O)n1N1CCCCC1]

The synthetic route is as follows:

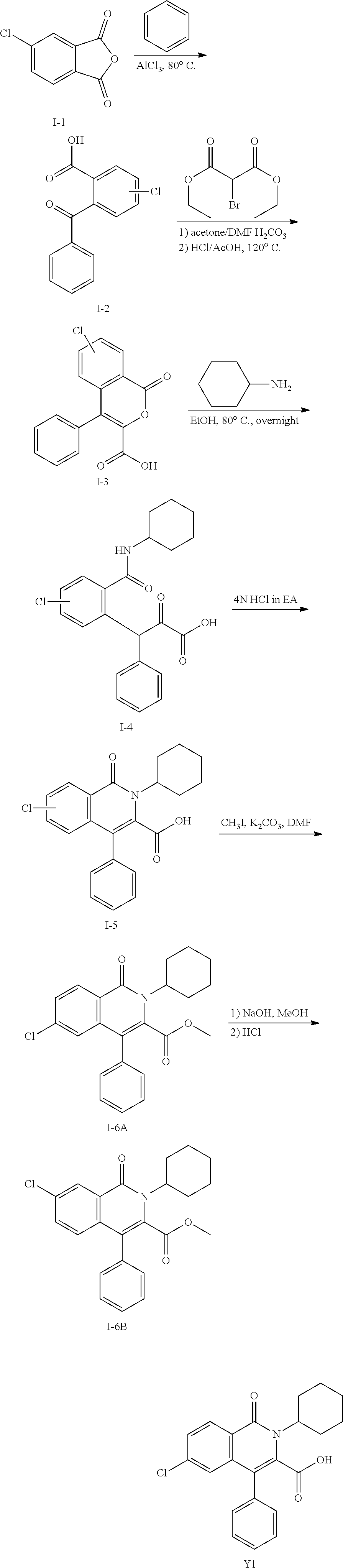

I-1 (5.0 g, 27.29 mmol) was added to an eggplant-shaped bottle, followed by adding benzene (20 ml) and aluminum chloride (7.3 g, 54.78 mmol). The reaction was carried out at room temperature for 45 min under argon protection, followed by reflux reaction for 2 h. The reaction solution was poured into ice water, and IN dilute hydrochloric acid (50 ml) was added and the mixture was stirred at room temperature for 0.5 h. The reaction solution was separated into an organic layer and an aqueous phase and the aqueous phase was extracted with ethyl acetate and then combined with the previous organic phase. The organic phase was then washed with saturated sodium chloride, dried over anhydrous sodium sulfate, and dried off the solvent by rotary evaporation to obtain a crude product. Toluene was added to the crude product, and 5 g of I-2 was obtained by recrystallization with a yield of 70.03%. ESI-MS: 259.0 [M−H]⁻.

I-2 (4.7 g, 18.03 mmol) was dissolved in acetone (30 ml), and potassium carbonate (2.49 g, 18.03 mmol) was then added and a large amount of solid precipitated. Diethyl 2-bromomalonate (5.17 g, 21.64 mmol) was dissolved in DMF (10 ml) and added to the above solution. The reaction was carried out at room temperature for 20 h. After the acetone was removed by rotary evaporation, the reaction solution was poured into water, and extracted with ethyl acetate three times, and the extract was washed with saturated sodium chloride, and dried over anhydrous sodium sulfate. After rotary evaporation, acetic acid (30 ml) and concentrated hydrochloric acid (30 ml) with equal volume were added, and the reflux reaction was carried out at 120° C. overnight. After the solvent was removed by rotary evaporation, 3.1 g of I-3 was obtained by slurrying with petroleum ether and ethyl acetate with a yield of 57.8%. ESI-MS: 299.0 [M−H]⁻.

I-3 (0.5 g, 1.66 mmol) and cyclohexylamine (2.47 g, 24.94 mmol) were added to a sealed tube. After adding ethanol (6 ml), the reflux reaction was carried out at 85° C. overnight. The next day, it was confirmed by LC-MS monitoring that the main product was I-4. After the solvent was removed by rotary evaporation, 4N of HCl solution in ethyl acetate (10 ml) was added to the sealed tube and it was reacted for 12 hours. The next day, a white solid precipitated. LC-MS monitoring showed that it was the target product I-5, 600 mg, yield 90.24%. ESI-MS: 380.1 [M−H]⁻. I-5 is a mixture of positional isomers, so it was separated and purified by esterification with carboxylic acid.

I-5 (600 mg, 1.57 mmol) was dissolved in DMF (6 mL), iodomethane (446 mg, 3.14 mmol) and potassium carbonate (434 mg, 3.14 mmol) were added, and the reaction was carried out at room temperature for 2 h. After the reaction was completed, the reaction solution was poured into water, extracted with ethyl acetate 3 times, and the extract was washed with saturated sodium chloride, dried over anhydrous sodium sulfate, and dried off the solvent by rotary evaporation to obtain a crude product, which was then purified by silica gel column chromatography to separate I-6A (with high polarity) and I-6B (with low polarity), obtaining I-6A 340 mg with a yield of 54.66%. ESI-MS: 380.1 [M−H]⁻. ¹H NMR (400 MHZ, Chloroform-d) δ8.38 (d, J=8.6 Hz, 1H), 7.49-7.41 (m, 4H), 7.31-7.26 (m, 2H), 7.10 (s, 1H), 3.65-3.55 (m, 1H), 3.51 (s, 3H), 2.74-2.65 (m, 2H), 1.90-1.86 (m, 4H), 1.35-1.23 (m, 3H).

I-6 (340 mg, 0.858 mmol) was dissolved in methanol (5 ml), 5N sodium hydroxide solution (5 ml) was added, and the mixture was reacted with heating at 70° C. overnight. The next day, after the reaction was completed, the methanol in the reaction product was evaporated off, and then the reaction solution was adjusted to pH 5 under ice bath conditions, extracted twice with ethyl acetate, and the extract was washed with saturated sodium chloride, dried over anhydrous sodium sulfate, and dried off the solvent by rotary evaporation to obtain a crude product. 152 mg of the target product was obtained by silica gel column chromatography, with a yield of 46.35%. ESI-MS: 380.1 [M−H]⁻. ¹H NMR (400 MHZ, Chloroform-d) δ8.23 (d, J=8.6 Hz, 1H), 7.45-7.37 (m, 4H), 7.34-7.31 (m, 2H), 7.10 (d, J=2.0 Hz, 1H), 3.73 (t, J=11.8 Hz, 1H), 2.67 (d, J=11.8 Hz, 2H), 1.87 (d, J=9.4 Hz, 4H), 1.24-1.15 (m, 2H).

### EXAMPLE 2

### 6-Chloro-2-(cyclohexylmethyl)-1-oxo-4-phenyl-1,2-dihydroisoquinoline-3-carboxylic Acid (Y2)

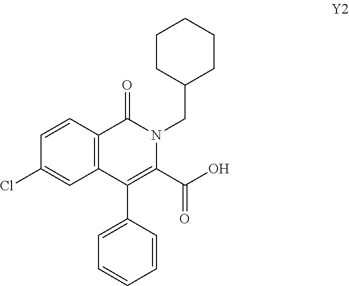 [SMILES: O=C(O)c1c(-c2ccccc2)c2cc(Cl)ccc2c(=O)n1CC1CCCCC1]

132 mg of target product was prepared with reference to the synthesis method in Example 1 with a yield of 34.45%. ESI-MS: 394.1 [M−H]⁻. ¹H NMR (400 MHZ, Chloroform-d) δ8.31 (d, J=8.6 Hz, 1H), 7.46-7.40 (m, 4H), 7.34-7.31 (m, 2H), 7.14 (d, J=2.0 Hz, 1H), 3.98 (d, J=7.3 Hz, 2H), 1.90-1.85 (m, 1H), 1.71-1.66 (m, 2H), 1.63-1.59 (m, 2H), 1.13 (d, J=8.9 Hz, 2H), 1.03-0.94 (d, J=11.7 Hz, 2H).

### EXAMPLE 3

### 6-Chloro-2-cyclopentyl-1-oxo-4-phenyl-1,2-dihydroisoquinoline-3-carboxylic Acid (Y3)

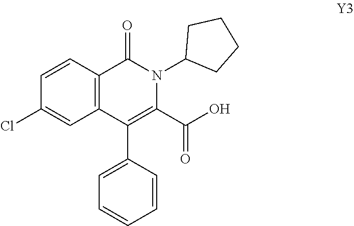 [SMILES: O=C(O)c1c(-c2ccccc2)c2cc(Cl)ccc2c(=O)n1C1CCCC1]

85 mg of target product was prepared with reference to the synthesis method in Example 1with a yield of 23.16%. ESI-MS: 366.1 [M−H]⁻. ¹H NMR (400 MHZ, DMSO-d6) δ8.28 (d, J=8.6 Hz, 1H), 7.58 (d, J=10.3 Hz, 1H), 7.50-7.43 (m, 3H), 7.34 (d, J=7.4 Hz, 2H), 6.90 (s, 1H), 4.26-4.20 (m, 1H), 2.35-2.27 (m, 2H), 2.01-1.92 (m, 2H), 1.89-1.81 (m 2H), 1.60-1.53 (m, 2H).

### EXAMPLE 4

### 6-Chloro-1-oxo-4-phenyl-2-(tetrahydro-2H-pyran-4-yl)-1,2-dihydroisoquinoline-3-carboxylic Acid (Y4)

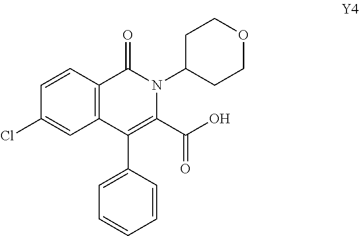 [SMILES: O=C(O)c1c(-c2ccccc2)c2cc(Cl)ccc2c(=O)n1C1CCOCC1]

35 mg of target product was prepared with reference to the synthesis method in Example 1 with a yield of 9.14%. ESI-MS: 382.1 [M−H]⁻. ¹H NMR (400 MHZ, DMSO-d6) δ8.29 (d, J=8.0 Hz, 1H), 7.59 (d, J=8.7 Hz, 1H), 7.50-7.43 (m, 3H), 7.33 (d, J=8.1 Hz, 2H), 6.88 (s, 1H), 3.98-3.90 (m, 3H), 3.25 (d, J=11.9 Hz, 2H), 2.96-2.86 (m, 2H), 1.66 (d, J=16.0 Hz, 2H).

### EXAMPLE 5

### 6-Chloro-2-cyclopropyl-1-oxo-4-phenyl-1,2-dihydroisoquinoline-3-carboxylic Acid (Y5)

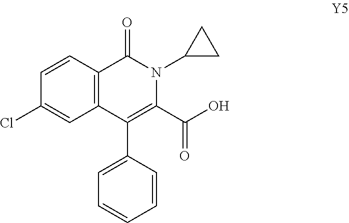 [SMILES: O=C(O)c1c(-c2ccccc2)c2cc(Cl)ccc2c(=O)n1C1CC1]

109 mg of target product was prepared with reference to the synthesis method in Example 1 with a yield of 19.29%. ESI-MS: 338.1 [M−H]⁻. ¹H NMR (400 MHz, Chloroform-d) δ8.35 (d, J=8.6 Hz, 1H), 7.47-7.43 (m, 4H), 7.33-7.28 (m, 2H), 7.10 (s, 1H), 3.21-3.17 (m, 1H), 1.09-1.06 (m, 2H), 0.95-0.93 (m, 2H).

### EXAMPLE 6

### 6-Chloro-2-cyclobutyl-1-oxo-4-phenyl-1,2-dihydroisoquinoline-3-carboxylic Acid (Y6)

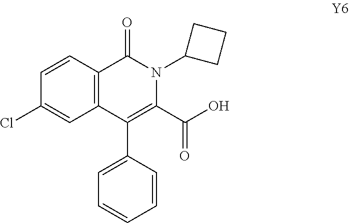 [SMILES: O=C(O)c1c(-c2ccccc2)c2cc(Cl)ccc2c(=O)n1C1CCC1]

132 mg of target product was prepared with reference to the synthesis method in Example 1 with a yield of 22.44%. ESI-MS: 352.1 [M−H]⁻. ¹H NMR (400 MHZ, Chloroform-d) δ8.29 (d, J=8.6 Hz, 1H), 7.46-7.41 (m, 4H), 7.30-7.27 (m, 2H), 7.08 (s, 1H), 4.65-4.57 (m, 1H), 2.72-2.62 (m,2H), 2.49-2.41 (m, 2H), 1.90-1.79 (m, 1H), 1.78-1.69 (m, 1H).

### EXAMPLE 7

### 6-Chloro-2-cycloheptyl-1-oxo-4-phenyl-1,2-dihydroisoquinoline-3-carboxylic Acid (Y7)

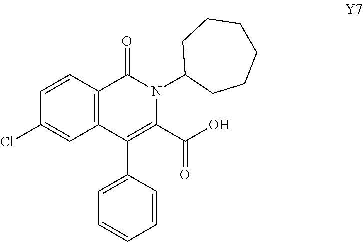 [SMILES: O=C(O)c1c(-c2ccccc2)c2cc(Cl)ccc2c(=O)n1C1CCCCCC1]

75 mg of target product was prepared with reference to the synthesis method in Example 1with a yield of 11.39%. ESI-MS: 394.1 [M−H]⁻. ¹H NMR (400 MHZ, Chloroform-d) δ8.26 (d, J=8.7 Hz, 1H), 7.45-7.43 (m, 3H), 7.40 (dd, J=8.7, 1.9 Hz, 1H), 7.36-7.32 (m, 2H), 7.10 (d, J=2.0 Hz,1H), 3.87 (s, 1H), 2.62 (s, 2H),2.00-1.93 (m, 2H), 1.85-1.76 (m, 2H), 1.66-1.54 (m,4H), 1.46-1.39 (m, 2H).

### EXAMPLE 8

### 6-Chloro-2-(4-chlorophenyl)-1-oxo-4-phenyl-1,2-dihydroisoquinoline-3-carboxylic Acid (Y8)

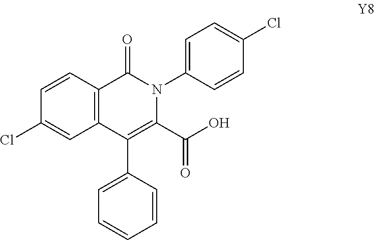 [SMILES: O=C(O)c1c(-c2ccccc2)c2cc(Cl)ccc2c(=O)n1-c1ccc(Cl)cc1]

31 mg of target product was prepared with reference to the synthesis method in Example 1 with a yield of 24.66%. ESI-MS: 408.1 [M−H]⁻. ¹H NMR (400 MHZ, Chloroform-d) δ8.25 (d, J=8.6 Hz, 1H), 7.52-7.41 (m, 4H), 7.34 (d, J=8.5 Hz, 2H), 7.29 (d, J=6.7 Hz, 2H), 7.21 (d, J=7.6 Hz,3H).

### EXAMPLE 9

### 6-Chloro-1-oxo-4-phenyl-2-(piperidin-1-yl)-1,2-dihydroisoquinoline-3-carboxylic Acid (Y9)

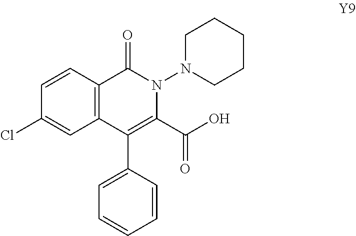 [SMILES: O=C(O)c1c(-c2ccccc2)c2cc(Cl)ccc2c(=O)n1N1CCCCC1]

20 mg of target product was prepared with reference to the synthesis method in Example 1 with a yield of 18.85%. ESI-MS: 381.1 [M−H]⁻. ¹H NMR (400 MHZ, Chloroform-d) δ8.37 (d, J=8.6 Hz, 1H), 7.47-7.40 (m, 4H), 7.36-7.34 (m, 2H), 7.20 (s, 1H), 3.90 (t, J=11.7 Hz, 2H), 3.15 (d, J=10.8 Hz, 2H), 1.75-1.63 (m, 3H), 1.44-1.35 (m, 2H).

### EXAMPLE 10

### 6-Bromo-2-cyclopentyl-1-oxo-4-phenyl-1,2-dihydroisoquinoline-3-carboxylic Acid (Y10)

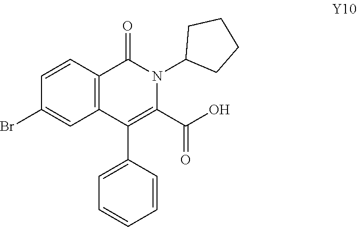 [SMILES: O=C(O)c1c(-c2ccccc2)c2cc(Br)ccc2c(=O)n1C1CCCC1]

121 mg of target product was prepared with reference to the synthesis method in Example 1 with a yield of 24.29%. ESI-MS: 410.1 [M−H]⁻. ¹H NMR (400 MHZ, Chloroform-d) δ8.21 (d, J=8.6 Hz, 1H), 7.57 (d, J=8.7 Hz, 1H), 7.47-7.42 (m, 3H), 7.35-7.31 (m, 2H), 7.27 (s, 1H), 4.30-4.21 (m,1H), 2.49-2.41 (m, 2H), 2.08-2.02 (m, 2H), 1.99-1.89 (m, 2H), 1.64-1.57 (m,2H).

### EXAMPLE 11

### 6,7-Dichloro-2-cyclopentyl-1-oxo-4-phenyl-1,2-dihydroisoquinoline-3-carboxylic Acid (Y11)

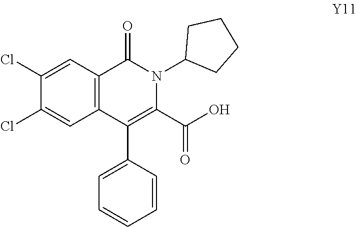 [SMILES: O=C(O)c1c(-c2ccccc2)c2cc(Cl)c(Cl)cc2c(=O)n1C1CCCC1]

240 mg of target product was prepared with reference to the synthesis method in Example 1with a yield of 39.99%. ESI-MS: 400.1 [M−H]⁻. ¹H NMR (400 MHZ, Chloroform-d) δ8.50 (s, 1H), 7.48-7.43 (m, 3H), 7.34-7.31 (m, 2H), 7.21 (s, 1H), 4.29-4.20 (m, 1H), 2.48-2.40 (m, 2H), 2.10-2.03 (m, 2H), 1.99-1.91 (m, 2H), 1.62-1.58 (m, 2H).

### EXAMPLE 12

### 2-Cyclopentyl-1-oxo-4-phenyl-1,2-dihydroisoquinoline-3-carboxylic Acid (Y12)

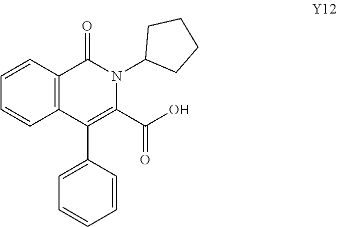 [SMILES: O=C(O)c1c(-c2ccccc2)c2ccccc2c(=O)n1C1CCCC1]

580 mg of target product was prepared with reference to the synthesis method in Example 1with a yield of 77.20%. ESI-MS: 332.1 [M−H]⁻. ¹H NMR (400 MHZ, Chloroform-d) δ8.32 (d, J=8.0 Hz, 1H), 7.55 (t, J=7.6 Hz, 1H), 7.47 (t, J=8.0 Hz, 1H), 7.41-7.38 m, 3H), 7.37-7.34 (m, 2H), 7.17 (d, J=8.0 Hz, 1H), 4.36-4.26 (m, 1H), 2.49-2.39 (m, 2H), 2.09-2.02 (m, 2H), 1.98-1.90 (m, 2H), 1.58-1.54 (m, 2H).

### EXAMPLE 13

### 2-Cyclopentyl-6-fluoro-1-oxo-4-phenyl-1,2-dihydroisoquinoline-3-carboxylic Acid (Y13)

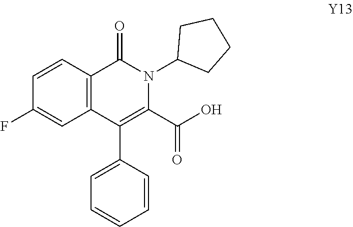 [SMILES: O=C(O)c1c(-c2ccccc2)c2cc(F)ccc2c(=O)n1C1CCCC1]

136 mg of target product was prepared with reference to the synthesis method in Example 1with a yield of 22.00%. ESI-MS: 350.1 [M−H]⁻. ¹H NMR (400 MHZ, Chloroform-d) δ8.31-8.25 (m, 1H), 7.43-7.39 (m, 3H), 7.33 (dd, J=6.6, 3.0 Hz, 2H), 7.14 (td, J=8.5, 2.5 Hz, 1H), 6.80 (dd, J=9.9, 2.5 Hz, 1H), 4.35-4.29 (m, 1H), 2.44-2.37 (m, 2H), 2.06-1.91 (m, 4H), 1.62-1.50 (m, 2H).

### EXAMPLE 14

### 2-Cyclopentyl-6-methyl-1-oxo-4-phenyl-1,2-dihydroisoquinoline-3-carboxylic Acid (Y14)

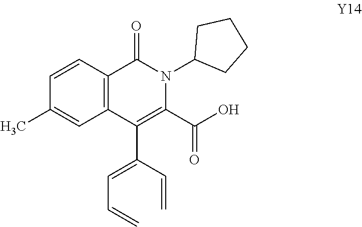 [SMILES: C=C/C=C(\C=C)c1c(C(=O)O)n(C2CCCC2)c(=O)c2ccc(C)cc12]

116 mg of target product was prepared with reference to the synthesis method in Example 1 5 with a yield of 29.00%. ESI-MS: 346.1 [M−H]⁻. ¹H NMR (400 MHZ, Chloroform-d) δ8.19 (d, J=8.3 Hz, 1H), 7.44-7.39 (q, J=3.6 Hz, 3H), 7.37-7.32 (m, 2H), 7.28 (d, J=8.9 Hz, 1H), 6.92 (s, 1H), 4.34-4.24 (m, 1H), 2.44-2.38 (m, 2H), 2.35 (s, 3H), 2.04-2.00 (m, 2H), 1.97-1.89 (m, 2H), 1.59-1.50 (m, 2H).

### EXAMPLE 15

### 10 6-Chloro-2-cyclopentyl-4-(4-fluorophenyl)-1-oxo-1,2-dihydroisoquinoline-3-carboxylic Acid (Y15)

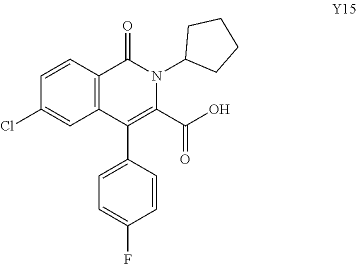 [SMILES: O=C(O)c1c(-c2ccc(F)cc2)c2cc(Cl)ccc2c(=O)n1C1CCCC1]

The synthesis route is as follows:

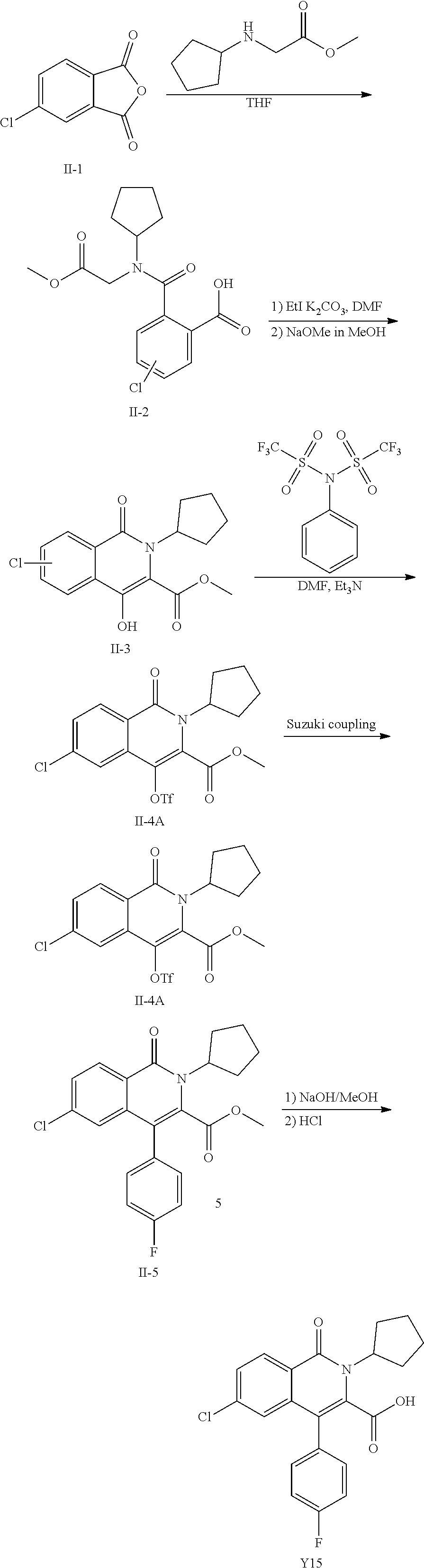

Compound II-1 (4.40 g, 24.10 mmol) was dissolved in tetrahydrofuran solution (20 ml), and then methyl N-cyclopentylglycinate (4.17 g, 26.51 mmol) was dissolved in an appropriate amount of tetrahydrofuran solution and added to the above reaction solution. The mixture was stirred at room temperature for 1 h. After the reaction was completed as monitored by LC-MS, the reaction solution was poured into water, and the aqueous layer was extracted with ethyl acetate for 3 times. The extract was washed with saturated sodium chloride, dried over anhydrous sodium sulfate, and dried off the solvent by rotary evaporation to obtain 7.0 g of the product. The product was directly used in the next step without purification, with a yield of 85.48%. ESI-MS: 340.1 [M+H]⁺.

Compound II-2 (7.0 g, 20.60 mmol) was dissolved in DMF (15 ml), and then iodoethane (4.82 g, 30.90 mmol) and potassium carbonate (8.54 g, 61.81 mmol) were added. The reaction solution was reacted at room temperature for three hours. After the reaction was completed as monitored by LC-MS, the reaction solution was poured into water, the aqueous layer was extracted with ethyl acetate three times, and then the extract was washed with saturated sodium chloride, dried over anhydrous sodium sulfate, and dried off the solvent by rotary evaporation. Methanol (30 ml) and sodium methoxide (7.6 ml, 5 mol/L sodium methoxide/methanol solution) were added to the bottle, and reacted at room temperature for 2 hours. After the reaction was completed as monitored by LC-MS, the pH of the reaction solution was adjusted to weak acidity with 1N hydrochloric acid solution, and the reaction solution was poured into water, extracted with ethyl acetate twice, and the extract was washed with saturated sodium chloride, dried over anhydrous sodium sulfate, and dried off the solvent by rotary evaporation to obtain 5.60 g of a crude product with a yield of 91.45%. ESI-MS: 322.1 [M+H]⁺.

II-3 (2.0 g, 6.22 mmol) was dissolved in DMF (10 ml), triethylamine (2.59 ml, 18.65 mmol) and 1,1,1-trifluoro-N-phenyl-N-((trifluoromethyl) sulfonyl) methanesulfonamide (2.66 g, 7.46 mmol) were added in order, and the reaction was carried out at room temperature overnight. After the reaction was completed as monitored by LC-MS, the reaction solution was poured into water, washed with ethyl acetate, dried over anhydrous sodium sulfate, and dried off the solvent by rotary evaporation to obtain a crude product. Two positional isomers II-4A (high polarity) and II-4B (low polarity) could be successfully separated by silica gel column chromatography, and finally 1.6 g of II-4A was obtained, with a yield of 56.72%. ESI-MS: 454.0 [M+H]⁺. ¹H NMR (400 MHZ, Chloroform-d) δ8.34 (d, J=8.8 Hz, 1H), 7.73 (s, 1H), 7.58 (d, J=9.3 Hz, 1H), 4.13-4.04 (m, 1H), 4.03 (s, 3H), 2.43-2.35 (m, 2H), 2.11-2.04 (m, 2H), 1.97-1.90 (m, 2H), 1.63-1.57 (m, 2H).

II-4A (250 mg, 0.55 mmol), p-fluorophenylboronic acid (93 mg, 0.66 mmol), potassium carbonate (152 mg, 1.10 mmol) and PdCl₂(dppf).CH₂Cl₂ (27 mg, 0.027 mmol) were added to an eggplant-shaped bottle, followed by adding dioxane and water. The reaction was carried out at 100° C. overnight under argon protection. After the reaction was completed, the reaction solution was dried off the solvent by rotary evaporation, added with ethyl acetate, washed with saturated sodium chloride, dried over anhydrous sodium sulfate and dried off the solvent by rotary evaporation to obtain a crude product. The product II-5 (123 mg) was obtained by silica gel column chromatography with a yield of 55.84%. ESI-MS: 400.1 [M+H]⁺.

II-5 (123 mg, 0.307 mmol) was dissolved in methanol (5 ml), and 5N sodium hydroxide solution (5 ml) was added. The reaction was carried out at 70° C. overnight. After the reaction was completed the next day, the methanol solution in the reaction was dried off by rotary evaporation. Then, the reaction solution was adjusted to pH 5 under ice bath conditions, extracted twice with ethyl acetate, and the extract was washed with saturated sodium chloride, dried over anhydrous sodium sulfate, and dried off the solvent by rotary evaporation to obtain a crude product. 63 mg of the product was obtained by silica gel column chromatography, with a yield of 53.08%. ESI-MS: 384.1 [M−H]⁻. ¹H NMR (400 MHZ, Chloroform-d) δ8.24 (d, J=8.6 Hz, 1H), 7.44 (d, J=8.6 Hz, 1H), 7.35-7.32 (m, 2H), 7.17-7.09 (m, 3H), 4.34-4.26 (m, 1H), 2.46-2.39 (m, 2H), 2.07-2.00 (m, 2H), 1.97-1.92 (m, 2H), 1.64-1.56 (m, 2H).

### EXAMPLE 16

### 6-Chloro-2-cyclopentyl-4-(4-hydroxyphenyl)-1-oxo-1,2-dihydroisoquinoline-3-carboxylic Acid (Y16)

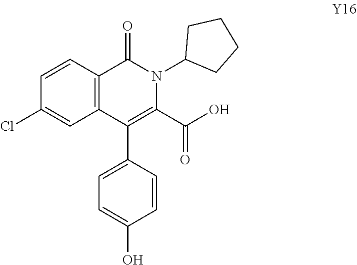 [SMILES: O=C(O)c1c(-c2ccc(O)cc2)c2cc(Cl)ccc2c(=O)n1C1CCCC1]

157 mg of target product was prepared with reference to the synthesis method in Example 15 with a yield of 65.10%. ESI-MS: 382.1 [M−H]⁻. ¹H NMR (400 MHZ, DMSO-d6) δ12.01 (s, 1H), 9.71 (s, 1H), 8.27 (d, J=8.2 Hz, 1H), 7.58 (d, J=8.2 Hz, 1H), 7.12 (d, J=8.2 Hz, 2H), 6.98 (s, 1H), 6.85 (d, J=8.3 Hz, 2H), 4.27-4.18 (m, 1H), 2.34-2.27 (m, 2H), 1.98-1.95 (m, 2H), 1.87-1.84 (m, 2H), 1.61-1.52 (d, J=8.7 Hz, 2H).

### EXAMPLE 17

### 6-Chloro-2-cyclopentyl-4-(4-(hydroxymethyl) phenyl)-1-oxo-1,2-dihydroisoquinoline-3-carboxylic Acid (Y17)

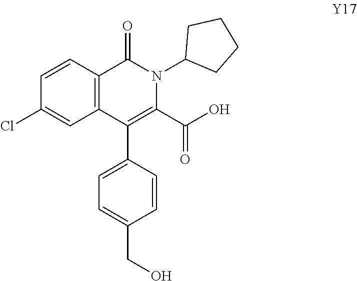 [SMILES: O=C(O)c1c(-c2ccc(CO)cc2)c2cc(Cl)ccc2c(=O)n1C1CCCC1]

83 mg of target product was prepared with reference to the synthesis method in Example 1 with a yield of 52.08%. ESI-MS: 396.1 [M−H]⁻. ¹H NMR (400 MHZ, DMSO-d6) δ8.29 (d, J=8.6 Hz, 1H), 7.59 (d, J=7.0 Hz, 1H), 7.42 (d, J=7.8 Hz, 2H), 7.30 (d, J=7.9 Hz, 2H), 6.93 (s, 1H), 5.31 (s, 1H), 4.57 (s, 2H), 4.29-4.21 (m, 1H), 2.35-2.28 (m, 2H), 1.99-1.93 (m, 2H), 1.87-1.82 (m, 2H), 1.58-1.54 (m, 2H).

### EXAMPLE 18

### 6-Chloro-4-(3-chloro-4-fluorophenyl)-2-cyclopentyl-1-oxo-1,2-dihydroisoquinoline-3-carboxylic Acid (Y18)

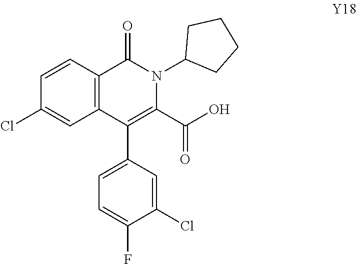 [SMILES: O=C(O)c1c(-c2ccc(F)c(Cl)c2)c2cc(Cl)ccc2c(=O)n1C1CCCC1]

221 mg of target product was prepared with reference to the synthesis method in Example 1 with a yield of 81.56%. ESI-MS: 418.1 [M−H]⁻. ¹H NMR (400 MHZ, Chloroform-d) δ8.29 (d, J=10.6 Hz, 1H), 7.44 (t, J=10.4 Hz, 2H), 7.25-7.18 (m, 2H), 7.07 (s, 1H), 4.36-4.24 (m, 1H), 2.47-2.42 (m, 2H), 2.12-2.03 (m, 2H), 1.98 (s, 2H), 1.65-1.60 (m, 2H).

### EXAMPLE 19

### 6-Chloro-2-cyclopentyl-1-oxo-4-(4-(trifluoromethyl) phenyl)-1,2-dihydroisoquinoline-3-carboxylic Acid (Y19)

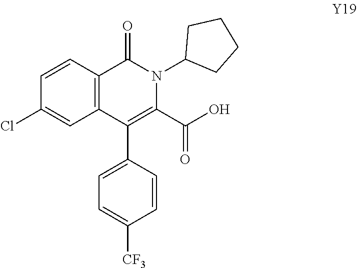 [SMILES: O=C(O)c1c(-c2ccc(C(F)(F)F)cc2)c2cc(Cl)ccc2c(=O)n1C1CCCC1]

231 mg of target product was prepared with reference to the synthesis method in Example 15 with a yield of 85.16%. ESI-MS: 434.1 [M−H]⁻. ¹H NMR (400 MHZ, DMSO-d6) δ8.31 (d, J=8.6 Hz, 1H), 7.87 (d, J=8.7 Hz, 2H), 7.63-7.59 (m, 3H), 6.91 (s, 1H), 4.30-4.22 (m, 1H), 2.36-2.28 (m, 2H), 2.02-1.94 (m, 2H), 1.91-1.84 (m, 2H), 1.62-1.56 (m, 2H).

### EXAMPLE 20

### 6-Chloro-2-cyclopentyl-4-(2,3-dihydrobenzofuran-5-yl)-1-oxo-1,2-dihydroisoquinoline-3-carboxylic Acid (Y20)

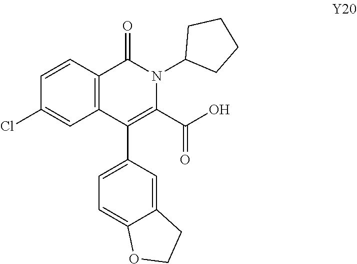 [SMILES: O=C(O)c1c(-c2ccc3c(c2)CCO3)c2cc(Cl)ccc2c(=O)n1C1CCCC1]

185 mg of target product was prepared with reference to the synthesis method in Example 15 with a yield of 83.19%. ESI-MS: 408.1 [M−H]⁻. ¹H NMR (600 MHZ, Chloroform-d) δ8.32 (d, J=8.6 Hz, 1H), 7.45 (d, J=8.7 Hz, 1H), 7.19 (s, 1H), 7.16 (s, 1H), 7.08 (d, J=8.1 Hz, 1H), 6.84 (d, J=7.0 Hz, 1H), 4.71-4.63 (m, 2H), 4.32-4.26 (m, 1H), 3.32-3.21 (m, 2H), 2.52-2.47 (m 2H), 2.12-2.07 (m, 2H), 2.04-1.94 (m, 2H), 1.66-1.59 (m, 2H).

### EXAMPLE 21

### 6-Chloro-2-cyclopentyl-1-oxo-4-(pyridin-3-yl)-1,2-dihydroisoquinoline-3-carboxylic Acid (Y21)

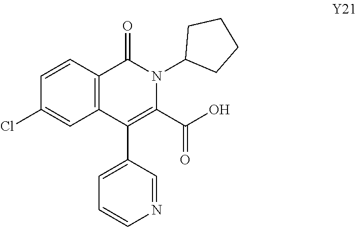 [SMILES: O=C(O)c1c(-c2cccnc2)c2cc(Cl)ccc2c(=O)n1C1CCCC1]

137 mg of target product was prepared with reference to the synthesis method in Example 15 with a yield of 64.64%. ESI-MS: 367.1 [M−H]⁻. ¹H NMR (600 MHZ, DMSO-d6) δ8.59 (d, J=4.8 Hz, 1H), 8.52 (s, 1H), 8.26 (d, J=8.6 Hz, 1H), 7.78 (d, J=7.7 Hz, 1H), 7.49-7.45 (m, 2H), 6.86 (s, 1H), 4.52-4.47 (m, 1H), 2.33-2.28 (m, 2H), 1.98-1.93 (m, 2H), 1.83-1.78 (m, 2H), 1.54-1.50 (m, 2H).

### EXAMPLE 22

### 6-Chloro-2-cyclopentyl-4-(1,3-dimethyl-1H-pyrazol-4-yl)-1-oxo-1,2-dihydroisoquinoline-3-carboxylic Acid (Y22)

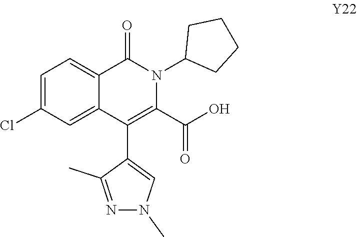 [SMILES: Cc1nn(C)cc1-c1c(C(=O)O)n(C2CCCC2)c(=O)c2ccc(Cl)cc12]

160 mg of target product was prepared with reference to the synthesis method in Example 15 with a yield of 52.31%. ESI-MS: 384.1 [M−H]⁻. ¹H NMR (400 MHZ, Chloroform-d) δ8.37 (d, J=8.0 Hz, 1H), 7.45 (d, J=8.0 Hz, 1H), 7.37 (s, 1H), 7.13 (s, 1H), 2.56-2.46 (m, 1H),3.84 (s, 3H), 2.56-2.46 (m, 2H), 2.11 (s, 2H), 2.06 (s, 3H), 2.01 (s, 2H), 1.68-1.64 (m, 2H).

### Pharmacological Activity Experiment Example

### EXAMPLE 23

### Activity Test at Molecular Level

We first tested the inhibition rate of the compounds prepared by the present invention on FABP4 at 25 μM. Considering that inhibition of FABP3 would cause myocardial function damage and bring potential safety issues, we tested the inhibitory activity of some compounds on FABP3. The activity test is illustrated by the example of the FABP4 activity test system as below.

Experimental principle and method: Once the free non-covalent fluorescent probe ANS binds to FABP4, the ANS fluorescence intensity will increase and its spectrum will be blue shifted. In this experiment, the inhibitory effect of the compound on FABP4 was evaluated by measuring the change in the ANS fluorescence signal value. For the FABP4 inhibitory activity test, the ANS substrate competition method was used, and we used the method of Kane and Bernlohr (E. Marr, M. Tardie, M. Carty, T. Brown Phillips, I. K. Wang, W. Soeller, X. Qiu, G. Karam, Expression, purification, crystallization and structure of human adipocyte lipid-binding protein (aP2). Acta Crystallogr., Sect. F: Struct. Biol. Cryst. Commun. 62 (2006) 1058-1060.). Human FABP4 with a His tag was expressed in the BL21 (DE3) strain and then purified using a Ni column with a His tag to generate the protein. In the detection system, the concentration of 1,8-ANS substrate was 10 μM, the final concentration of FABP4 was 10 μM, and then the compound prepared by the present invention was added at the required concentration for incubation for 3 minutes, and finally the fluorescence signal was detected at an excitation wavelength (EX) of 370 nm/emission wavelength (EM) of 470 nm. The inhibition rate (%) of the test sample on FABP4 was calculated according to the fluorescence absorption value of the system. The inhibition rate (%) was calculated according to the following equation:

$$Inhibition rate ( \% ) = [ 1 - ( {F}_{X} = {F}_{background} ) / ( {F}_{0} \% - {F}_{background} ) ] * 100 \%$$

In the above equation:

F_{X} represents the fluorescence value (fluorescence, F) of the system measured in the presence of compound x;

F_{background} represents the fluorescence value of the fluorescent substrate ANS;

F₀% represents the fluorescence value of the system when the inhibition rate is 0%, that is, when no compound was added.

| Compound | FABP4@25 μM | FABP5@25 μM | FABP3@25 μM |
|---|---|---|---|
| Y1 | 70% | 50% | 29% |
| Y2 | 93% | 73% | 48% |
| Y3 | 93% | 68% | 36% |
| Y4 | 71% | 50% | 14% |
| Y5 | 88% | 43% | 82% |
| Y6 | 94% | 60% | 81% |
| Y7 | 78% | 57% | 18% |
| Y8 | 35% | 1% | / |
| Y9 | 95% | 72% | 59% |
| Y10 | 95% | 58% | 57% |
| Y11 | 77% | 41% | 48% |
| Y12 | 58% | 25% | / |
| Y13 | 89% | 43% | 11% |
| Y14 | 90% | 55% | 26% |
| Y15 | 91% | 52% | 27% |
| Y16 | 92% | 52% | 28% |
| Y17 | 1% | 14% | / |
| Y18 | 91% | 46% | 16% |
| Y19 | 14% | 22% | 3% |
| Y20 | 32% | 25% | / |
| Y21 | 76% | 31% | 9% |
| Y22 | 36% | 36% | 1% |

Note:
“/” means not tested.

### EXAMPLE 24

In this example, the lipolysis inhibitory activity of the compounds prepared in the present invention on differentiated mature 3T3-L1 preadipocytes was measured.

5 In adipocytes, FABP4 plays the role of a fatty acid chaperone, and gene knockout or drug inhibition of FABP4 can be used to inhibit lipolysis and promote lipid production in adipocytes. Therefore, the lipolysis experiment can be used as a confirmation indicator of the FABP4 inhibitory activity of the compound. In this experiment, the lipolysis activity of the compounds was evaluated by measuring the effect of the compounds prepared in the present 10 invention on the glycerol content of the supernatant of mature adipocytes.

Experimental materials: 3T3-L1 preadipocytes; calf serum, fetal bovine serum, high glucose H-DMEM culture medium, 3-isobutyl-1-methylxanthine (IBMX), dexamethasone, insulin, Forskolin, glycerol determination kit, BCA protein concentration determination kit.

### Experimental Method

1. 3T3-L1 preadipocytes were inoculated on culture plates and cultured in high-glucose DMEM containing 10% calf serum in a 37° C., 5% CO₂ incubator;

2. After the cells were fully grown, contact inhibition was performed for two days;

3. The cells were cultured in high-glucose DMEM containing 10% fetal bovine serum with a final concentration of 0.5 mmol/L IBMX, a final concentration of 1 umol/L dexamethasone and a final concentration of 5 μg/ml insulin for 48 h;

4. After 48 h, the medium was replaced with high-glucose DMEM containing 10% fetal bovine serum with a final concentration of 5 μg/ml insulin and the cells were cultured therein for another 48 h. The culture medium was changed every 2 days. After inducing differentiation for 8-12 days, more than 90% of 3T3-L1 cells became mature adipocyte phenotype;

5. Compounds were added for incubation for 24 hours, and the incubated cells were washed twice with Krebs Ringer HEPES buffer, and then stimulated with forskolin at a final concentration of 20 μM for two hours;

6. The cell supernatant was collected to measure the glycerol content, the intracellular protein concentration was measured by BCA method for calibration, and finally Graphpad Prism was used to analyze the results.

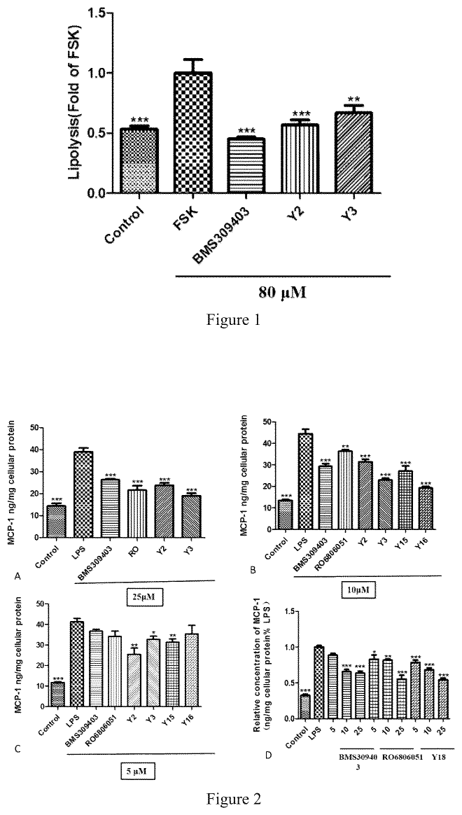

The cell results are shown in FIG. 1. Compounds Y2 and Y3 can significantly reduce the glycerol content of the supernatant under FSK (forskolin) stimulation at a concentration of 80μM.

### EXAMPLE 25

Effects of the compounds on monocyte chemoattractant factor-1 (MCP-1) and interleukin-6 (IL-6) in THP-1 monocyte-derived macrophage.

First, monocytic cell line THP-1 in logarithmic growth phase was plated on a 96-well plate at a cell density of 5*10⁵/ml; then 100 nM phorbol myristate ester (PMA) was added to induce the cells for 24 hours to differentiate into macrophages; then the cells were washed once or twice with phosphate buffered saline (PBS), and different concentrations of compounds (5, 10, 25 μM) were added for incubation for 18 hours; finally, after 6 hours of stimulation with 100 ng/ml lipopolysaccharide (LPS), the supernatant was collected and diluted to a certain multiple and the contents of MCP-1 and IL-6 were detected using enzyme-linked immunosorbent assay (ELISA), and cell protein was collected and the protein concentration was measured by BCA method for calibration.

As shown in FIG. 2, compounds Y2, Y3, Y15, Y16 and Y18 could reduce the secretion of inflammatory factor MCP-1 in THP-1 mononuclear macrophages at concentrations of 25 μM, 10 μM and 5 μM, and the activities of compounds Y2 and Y3 were better than those of the positive control FABP4 inhibitor BMS309403 and the lead compound RO6806051.

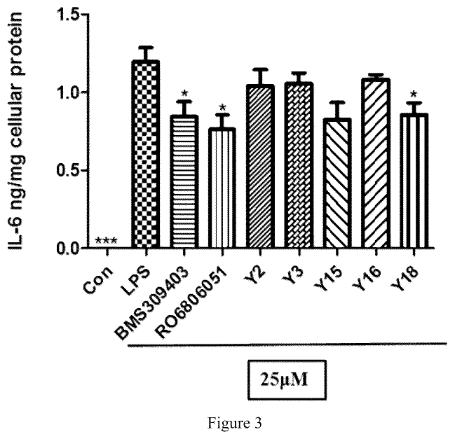

As shown in FIG. 3, compound Y18 could reduce the secretion of inflammatory factor IL-6 in THP-1 mononuclear macrophages at a concentration of 25 μM, and Y2, Y3, Y15 and Y18 had a tendency to reduce IL-6 secretion but with no statistical difference.

The above examples are only used to illustrate the technical solutions of the present invention, rather than to limit the present invention. Although the present invention has been described in detail with reference to the above examples, those skilled in the art should understand that they can still modify the technical solutions described in the above examples, or replace some of the technical features therein by equivalents. However, these modifications or replacements do not deviate the essence of the corresponding technical solutions from the spirit and scope of the technical solutions of the examples of the present invention.

1. An isoquinolinone compound represented by the general formula (I), a pharmaceutically acceptable salt, stereoisomer, prodrug molecule thereof or their mixture: 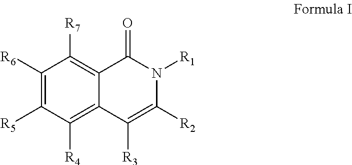
wherein,
R₁ is selected from the group consisting of substituted or unsubstituted phenyl, C₅-C₁₂ aromatic heterocyclyl containing oxygen, nitrogen and/or sulfur, C₃-C₈ cycloalkyl, and 4-7 membered heterocyclyl containing 1 to 3 heteroatoms of oxygen, nitrogen and/or sulfur; in the case where the R₁ group has a substituent, each substituent is independently selected from the group consisting of halogen, hydroxyl, hydroxymethyl, thiol, amino, cyano, nitro, carboxyl, ester group, trifluoromethyl, trifluoromethoxy, C₁-C₆ straight-chain or branched-chain alkyl, C₁-C₆ straight-chain or branched-chain haloalkyl, C₁-C₆ straight-chain or branched-chain alkoxy, C₁-C₆ straight-chain or branched-chain haloalkoxy, C₁-C₆ straight-chain or branched-chain alkylcarbonyloxy, and C₁-C₆ straight-chain or branched-chain hydroxyalkyl:
R₂ is selected from the group consisting of carboxyl, cyano, hydroxyl, sulfonyl hydroxide, sulfonamide group, amide group, tetrazole group, squaryl, phosphate group, hydroxamic acid group, and hydroxyisoxazole group:
R₃ is selected from the group consisting of substituted or unsubstituted phenyl: C₆-C₁₂ aryl: benzyl: C₅-C₇ aromatic heterocycle containing oxygen, nitrogen and sulfur: C₃-C₁₄ cycloalkyl: and 4-8 membered heterocyclyl: in the case where the R₃ group has a substituent (i.e. “substituted”), each substituent is independently selected from the group consisting of halogen, hydroxyl, hydroxymethyl, thiol, amino, cyano, nitro, carboxyl, ester group, trifluoromethyl, trifluoromethoxy, C₁-C₆ straight-chain alkyl or C₃-C₆ branched-chain alkyl, C₁-C₆ straight-chain haloalkyl or C₃-C₆ branched-chain haloalkyl, C₁-C₆ straight-chain alkoxy or C₃-C₆ branched-chain alkoxy, and C₁-C₆ straight-chain haloalkoxy:
R₄ to R₇ are each independently selected from the group consisting of hydrogen, halogen, hydroxyl, amino, cyano, thiol, trifluoromethyl, trifluoromethoxy, nitro, amide group, sulfonamide group, C₁-C₃ straight-chain or branched alkyl, and C₁-C₃ straight-chain or branched-chain alkoxy:

2. The isoquinolinone compound, the pharmaceutically acceptable salt, stereoisomer, prodrug molecule thereof or their mixture of claim 1, wherein,
R₁ is selected from the group consisting of C₃-C₈ cycloalkyl, 4-7 membered heterocyclyl containing 1 to 3 heteroatoms of oxygen, nitrogen and/or sulfur, and substituted or unsubstituted phenyl: in the case where the R₁ group has a substituent (i.e. “substituted”), each substituent is independently selected from the group consisting of halogen, hydroxyl, hydroxymethyl, thiol, amino, cyano, nitro, carboxyl, ester group, trifluoromethyl, trifluoromethoxy, C₁-C₃ straight-chain or branched-chain alkyl, and C₁-C₃ straight-chain or branched-chain alkoxy:
R₂ is selected from the group consisting of carboxyl, sulfonyl hydroxide, sulfonamide group, amide group, tetrazole group, squaryl, and hydroxyisoxazole group:
R₃ is selected from the group consisting of substituted or unsubstituted phenyl: C₅-C₇ aromatic heterocycle containing oxygen, nitrogen and sulfur: C₃-C₁₄ cycloalkyl; and 4-8 membered heterocyclyl: in the case where the R₃ group has a substituent (i.e. “substituted”), each substituent is independently selected from the group consisting of halogen, hydroxyl, hydroxymethyl, thiol, amino, cyano, nitro, carboxyl, ester group, trifluoromethyl, and trifluoromethoxy:
R₄ to R₇ are each independently selected from the group consisting of hydrogen, halogen, hydroxyl, amino, trifluoromethyl, trifluoromethoxy, nitro, and methoxy:
preferably, R₂ is —COOH, R₃ is phenyl, substituted phenyl (e.g., chlorophenyl, fluorophenyl, methylphenyl, benzyl, trifluoromethylphenyl) or aromatic heterocyclyl, R₄, R₆ and R₇ are independently hydrogen:
further preferably, R₁ is selected from the group consisting of cyclohexyl, methylcyclohexyl, cyclopentyl, tetrahydropyranyl, cyclopropyl, cyclobutyl, cycloheptyl, chlorophenyl, piperidinyl and cyclopentyl: R₂ is carboxyl: R₃ is selected from the group consisting of phenyl, fluorophenyl, hydroxyphenyl, hydroxymethylphenyl, chlorofluorophenyl, trifluoromethylphenyl, dihydrobenzofuranyl, pyridinyl, and dimethylpyrazolyl; R₄ and R₇ are independently H or they are simultaneously H, R₅ is Cl, Br or methyl; and R₆ is H or Cl.

3. The isoquinolinone compound, the pharmaceutically acceptable salt, stereoisomer, prodrug molecule thereof or their mixture of claim 1, wherein the compound is selected from the group consisting of the following compounds:

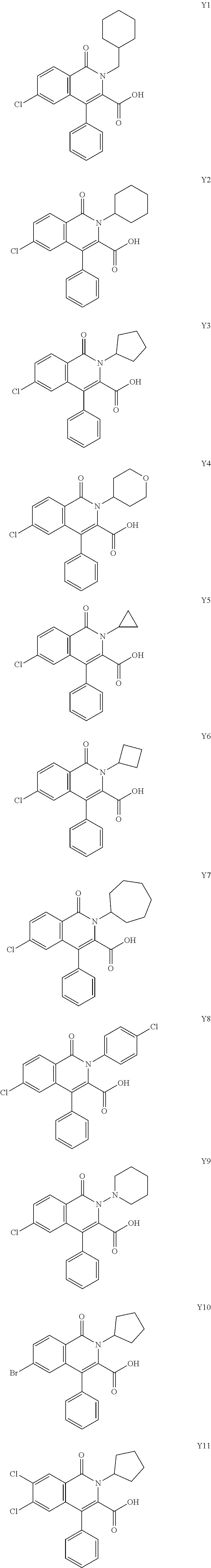

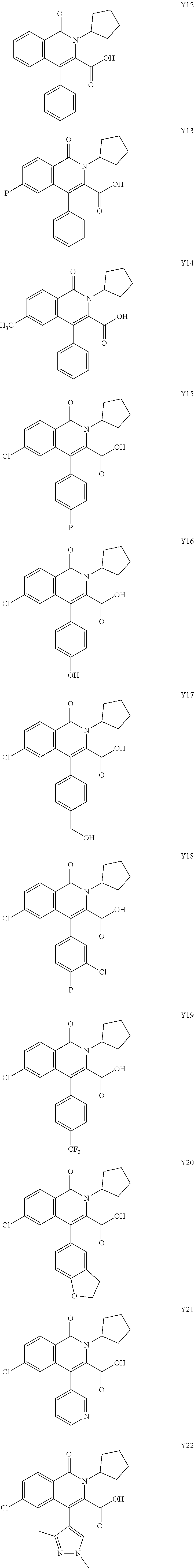

4. Use of the isoquinolinone compound, the pharmaceutically acceptable salt, stereoisomer, prodrug molecule thereof or their mixture of claim 1 in the preparation of a drug for preventing or treating FABP4/5-mediated diseases.

5. Use of claim 5, wherein, the FABP4/5-mediated diseases are metabolic diseases, cardiovascular and cerebrovascular diseases, inflammatory diseases, autoimmune diseases, organ fibrosis diseases, neurological damage diseases, secondary diseases caused by pathogen infection or tumors.

6. Use of claim 5, wherein, the metabolic disease is selected from one or more of insulin resistance, type 2 diabetes, atherosclerosis, hyperlipidemia and non-alcoholic fatty liver disease: the autoimmune disease is selected from one or more of psoriasis and rheumatoid arthritis; the acute inflammation is selected from one or more of sepsis, septicemia, acute kidney injury, acute lung injury, and acute liver injury: the cancer is selected from one or more of malignant melanoma, lung cancer, gastric cancer, breast cancer, colon cancer, bladder cancer, pancreatic cancer, lymphoma, leukemia, prostate cancer, ovarian cancer, liver cancer, and cervical cancer.

7. A pharmaceutical composition, which contains a therapeutically effective amount of the isoquinolinone compound, the pharmaceutically acceptable salt, stereoisomer, prodrug molecule thereof or their mixture of any one of claims 1 to 3.

8. The pharmaceutical composition of claim 7, wherein the isoquinolinone compound, the pharmaceutically acceptable salt, stereoisomer, prodrug molecule thereof or their mixture accounts for 20% to 99% of the total weight of the pharmaceutical composition.

9. The pharmaceutical composition of claim 7 or 8, wherein the composition further comprises one or more pharmaceutically acceptable carriers, odorants, flavoring agents, vehicles and/or diluents.

10. Use of the pharmaceutical composition of any one of claims 7 to 9 in the preparation of a drug for preventing and treating FABP4/5-mediated diseases, wherein the diseases are metabolic diseases, cardiovascular and cerebrovascular diseases, inflammatory diseases, autoimmune diseases, organ fibrosis diseases, neurological damage diseases, secondary diseases caused by pathogen infection or tumors.
